(19) United States
(12) Patent Application Publication
(10) Pub. No.: US 2025/0202598 A1
(43) Pub. Date: Jun. 19, 2025

(54) FREQUENCY MODULATED COMMUNICATION

(71) Applicant: Iota Biosciences, Inc., Alameda, CA (US)

(72) Inventors: Suraj GOWDA, Moraga, CA (US); Emma KRANICH, Delmar, NY (US); Albert YOU, Berkeley, CA (US)

(21) Appl. No.: 18/849,774

(22) PCT Filed: Mar. 23, 2023
(86) PCT No.: PCT/US2023/064885

§ 371 Date: Sep. 23, 2024

Related U.S. Application Data
(60) Provisional application No. 63/322,987, filed on Mar. 23, 2022.

(51) Int. Cl. H04B 11/00; H04B 1/713

(52) U.S. Cl. CPC H04B 11/00; H04B 1/713

(57) ABSTRACT

Methods and systems for low-power communication using ultrasonic waves are described herein. The methods may include receiving, at one or more ultrasonic transducers of an implantable device, ultrasonic waves transmitted by an interrogator; and emitting, from the one or more ultrasonic transducers of the implantable device, ultrasonic backscatter comprising encoded data, wherein the data is encoded into the ultrasonic backscatter by modulating a. frequency of the ultrasonic backscatter. The device may include an ultrasonic transducer configured to receive ultrasonic waves and emit ultrasonic backscatter; a switch configured to modulate a frequency of the emitted ultrasonic backscatter, and a circuit configured to operate the switch to encode data, in the emitted ultrasonic backscatter based on the frequency.

### PRIORITY

This application claims priority to U.S. Patent Application Ser. No. 63/322,987, filed Mar. 23, 2022, the entire contents of which are incorporated herein by reference.

### FIELD OF THE DISCLOSURE

Methods and systems for low power communication using ultrasonic backscatter communication is described herein. Also described are implantable devices that use such methods and systems.

### BACKGROUND OF THE DISCLOSURE

Backscatter ultrasonic communication is a low power method of communication for transmitting data. Such a low power method is preferable for communication between a device implantable in a patient and an external device (referred to herein as an interrogator). Typical communication between an interrogator and an implantable device can include emission of a backscattered ultrasonic pulse based on amplitude modulation at the implanted device. However, amplitude modulation is subject to noise issues (such as passive reflections from environmental structures) and thus low signal-to-noise (SNR) resulting in digital communication errors between the interrogator and the implantable device. Alternatives to amplitude modulated backscatter communications include active transmit and cancellation methods. However, as described further below, these alternatives can introduce significant disadvantages.

Active transmit generally relies on an internal battery to power signal emission out of the body. Active implants have expanded readable ranges, allow more variation in transmit information, and eliminate issues from passive reflections. Although active transmit avoids amplitude modulated issues, the introduction of a battery may increase the necessary size of the implant and introduce new challenges regarding battery recharging, safety, and cost.

Cancellation methods can be introduced to limit environmental interference passive reflections from environmental interference. Cancellation methods send a passive pulse to “learn” the reflective environment and then sends a calibrated active pulse to determine the modulation due to the implant. Using the difference of these two reflections helps to limit effects of environmental structures that may also reflect the ultrasound waves causing passive reflections. Although this limits issues due to passive reflections, these cancellation methods do not account for environmental interferences and changes that occur after the training pulses during the actual data transfers.

Therefore, challenges remain in overcoming passive reflections during ultrasonic backscatter communication between an interrogator and an implantable device.

### SUMMARY OF THE DISCLOSURE

Amplitude modulation can be used to encode data into the ultrasonic backscatter for sending information from the implantable device to an external device. However, as described in the previous section, communication based on amplitude modulation is subject to noise from passive reflections and other sources of reflective interference which can negatively affect communication. Thus, there remains a need for encoding data into the ultrasonic backscatter to enable low-power and low-noise communication between the implantable device and the interrogator.

Methods and systems for low-power communication using ultrasonic waves are described herein. The methods may include receiving, at one or more ultrasonic transducers of an implantable device, ultrasonic waves transmitted by an interrogator; and emitting, from the one or more ultrasonic transducers of the implantable device, ultrasonic backscatter comprising encoded data, wherein the data is encoded into the ultrasonic backscatter by modulating a frequency of the ultrasonic backscatter. The device may include an ultrasonic transducer configured to receive ultrasonic waves and emit ultrasonic backscatter; a switch configured to modulate a frequency of the emitted ultrasonic backscatter; and a circuit (which is optionally an integrated circuit) configured to operate the switch to encode data in the emitted ultrasonic backscatter based on the frequency.

A method for low-power communication using ultrasonic waves can include receiving, at one or more ultrasonic transducers of an implantable device, ultrasonic waves transmitted by an interrogator; and emitting, from the one or more ultrasonic transducers of the implantable device, ultrasonic backscatter comprising encoded data, wherein the data is encoded into the ultrasonic backscatter by modulating a frequency of the ultrasonic backscatter. The method may include selecting a switching frequency from a plurality of different switching frequencies to modulate the frequency of the ultrasonic backscatter, wherein the switching frequency is configured to encode the data into the ultrasonic backscatter. In some embodiments, each switching frequency is associated with a different predetermined bit pattern. Modulating the frequency of the ultrasonic backscatter may include switching a switch of the implantable device at the switching frequency.

In some implementations, each pre-determined bit pattern comprises two or more digital bits. In some implementations, pre-determined bit patterns associated with the plurality of different switching frequencies are gray coded.

The switching frequency may be based at least on instructions received from the interrogator via the ultrasonic waves.

The plurality of different switching frequencies may be selected based on at least an intensity of the ultrasonic backscatter at a given frequency.

The method may further include detecting sensor information from one or more sensors of the implantable device, wherein the data comprises one or more of sensor information, and the method comprising selecting the switching frequency from the plurality of different switching frequencies based at least on the sensor information. The sensor information may comprise, for example, one or more of a power level, pH, a temperature, a pressure, an electrophysiological pulse, and an analyte concentration. In some implementations, the analyte concentration comprises an oxygen level.

The method may include determining operating status information of the implantable device, wherein the data comprises determined operating status information, and the method can include selecting the switching frequency from the plurality of different switching frequencies based at least on the determined operating status information.

The method may include selecting a sequence of two or more switching frequencies from a plurality of different switching frequencies to encode the data, wherein each switching frequency is associated with a different predetermined bit pattern.

The method may further include decoding the ultrasonic backscatter received at the interrogator, wherein decoding the ultrasonic backscatter comprises analyzing an incoming frequency of the ultrasonic backscatter received at the interrogator to determine a computed switching frequency of a switch modulating the frequency of the ultrasonic backscatter, matching the computed switching frequency to a predetermined bit pattern, and recording the predetermined bit pattern as data transferred from the implantable device. In some implementations, decoding the backscatter comprises a bit error rate of up to 6%.

The method may include adjusting a parameter of the ultrasonic waves transmitted by the interrogator based on the ultrasonic backscatter received from the implantable device.

In some implementations, modulating the frequency of the ultrasonic backscatter may include switching a switch of the implantable device between an open state and a closed state at a switching frequency from a plurality of different switching frequencies. For example, the closed state may short the ultrasonic transducer. The open state may be a loaded configuration in which electrical energy is harvested from the ultrasonic waves and a maximum power is transferred from the ultrasonic waves to the implantable device to power the implantable device.

The method may include switching the switch of the implantable device between the open state and the closed state is configured to periodically (e.g., sinusoidally) vary acoustic energy reflected by the implantable device.

The method may include instructing the implantable device by the interrogator via the ultrasonic waves such that the implantable device executes one or more functions. For example, the one or more functions may include determining an average incident power from the ultrasonic waves.

The method may further include transmitting the ultrasonic waves from the interrogator.

The method may further include receiving the ultrasonic backscatter at the interrogator.

In some implementations of the method, the interrogator comprises one or more transducers for transmitting the ultrasonic waves and receiving the ultrasonic backscatter.

A device for communicating via ultrasonic waves can include: an ultrasonic transducer configured to receive ultrasonic waves and emit ultrasonic backscatter; a switch configured to modulate a frequency of the emitted ultrasonic backscatter; and a circuit (which is optionally an integrated circuit) configured to operate the switch to encode data in the emitted ultrasonic backscatter based on the frequency.

In some implementations of the device, the switch is switchable at a switching frequency from a plurality of different switching frequencies and the data encoded in the emitted ultrasonic backscatter is based on the switching frequency, wherein each switching frequency is associated with a different predetermined bit pattern. In some implementations, each pre-determined bit pattern comprises two or more digital bits. Pre-determined bit patterns associated with the plurality of different switching frequencies may be gray coded, in some implementations.

The interrogator may be configured to transmit the ultrasonic waves comprising instructions, and the switching frequency may be based at least on the instructions received from the interrogator via the ultrasonic waves.

In some implementations, the switching frequency is based at least on a relative maximum intensity of ultrasonic backscatter receivable at an interrogator.

The device may further include one or more sensors configured to detect information (e.g., sensory information), wherein the data comprises detected information, and the switching frequency from the plurality of different switching frequencies may be based at least on the detected information. The information may include, for example, a power level of the device. In some implementations, the one or more sensors comprises one or more of a pressure sensor, a pH sensor, a temperature sensor, and an analyte sensor.

In some implementations, the device may include a status indicator, and the switching frequency from the plurality of different switching frequencies is based at least on one or more statuses of the status indicator.

In some implementations, the switch is switchable in a sequence of two or more switching frequencies from a plurality of different switching frequencies to encode the data. Each switching frequency may be, for example, associated with a different predetermined bit pattern.

In some implementations, the interrogator is configured to receive the ultrasonic backscatter, decode the ultrasonic backscatter by analyzing an incoming frequency of the ultrasonic backscatter to determine a computed switching frequency of the switch, match the computed switching frequency to a predetermined bit pattern, and record the predetermined bit pattern as data transferred from the device. In some embodiments, the interrogator may analyze the ultrasonic backscatter at a bit rate error of up to 6% for decoding the ultrasonic backscatter.

In some implementations, the ultrasonic waves are transmitted by an interrogator and the interrogator is configured to adjust a parameter of the ultrasonic waves transmitted by the interrogator based on the ultrasonic backscatter received from the device.

In some implementations, the switch is switchable between an open state and a closed state at a switching frequency from a plurality of different switching frequencies. The closed state, for example, may short the one or more ultrasonic transducers of the device. In some implementations, the open state is a loaded configuration in which electrical energy is harvested from the ultrasonic waves and a maximum power is transferred from the ultrasonic waves to the device to power the device. In some implementations, switching the switch between the open state and the closed state is configured to periodically (e.g., sinusoidally) vary acoustic energy reflected by the device.

In some implementations of the device, the ultrasonic wave transmitted from an interrogator are configured to instruct the device to execute one or more functions. For example, the one or more functions may include a determination of average incident power from the ultrasonic waves.

In some implementations, the interrogator comprises one or more transducers configured to transmit the ultrasonic waves and receive the ultrasonic backscatter.

In some implementations, the device is an implantable device.

In some implementations, the device includes two or more electrodes that can emit an electrical pulse to a tissue. In some implementations, the device includes two or more electrodes that can detect an electrophysiological pulse.

Further described herein is a system for communicating via ultrasonic waves, that includes the device and the interrogator, which can include one or more ultrasonic transducers configured to receive the emitted ultrasonic backscatter and transmit the ultrasonic waves to the device; and a processor configured to decode the emitted ultrasonic backscatter.

### BRIEF DESCRIPTION OF THE DRAWINGS

The disclosure will now be described, by way of example only, with reference to the accompanying drawings, in which:

FIG. 1 shows an example of how a switch of the implantable device can be operated to modulate the frequency of the ultrasonic backscatter, according to some embodiments.

FIG. 2 shows an example of modulating a frequency of ultrasonic backscatter based on a frequency of the carrier signal and a switching frequency of a switch. The carrier signal is shown in the top panel and the switch state is shown in the second panel. The backscatter signal generated from mixing the carrier signal with the switch state is shown in the third panel.

FIG. 3 shows an example of envelope-extracted backscatter at different switching frequencies, represented by Frequency 0 and Frequency 1 of Table 1, according to some embodiments.

FIG. 4 shows a flow chart that describes a method 400 for detecting a signal at the interrogator, according to some embodiments.

FIG. 5 shows a plot of bit error rate (BER) for each transducer channel of an interrogator that includes sixteen channels, according to some embodiments.

FIG. 6 shows an exemplary plot of power levels read from frequency modulation based on piezoelectric voltage at the implantable device using the thresholds of Table 2, according to some embodiments.

FIG. 7 shows an example of ultrasonic backscatter magnitudes received at the interrogator versus the calculated switching frequency for a channel, according to some embodiments. As shown in this figure, different switching frequencies have higher or lower signal-to-noise, likely depending on the physical properties of the ultrasonic transducer (e.g., the piezoelectric crystal) used.

FIG. 8 shows an example flow diagram that describes a steering method for closed-loop power-delivery algorithm that utilizes the communication methods described herein, according to some embodiments.

FIG. 9 shows a flow diagram that describes a method 1000 for low-power communication using ultrasonic waves, according to some embodiments.

FIG. 10 shows an interrogator in communication with an implantable device, according to some embodiments.

### DETAILED DESCRIPTION OF EXEMPLARY EMBODIMENTS

Noise caused by passive reflections and other sources of reflective interference can alter accuracy and quality of data communication encoded by amplitude modulation of ultrasonic backscatter. Described herein are devices and systems that enable ultrasonic communication that is more protected against passive reflections and other sources of reflective interference. Further described are methods for using the devices and systems for low-power and low-noise ultrasonic communication. The devices and systems include an ultrasonic transducer configured to receive ultrasonic waves and emit ultrasonic backscatter and include a switch for modulating a frequency of the emitted ultrasonic backscatter. The switch can be operated to modulate a frequency of the ultrasonic backscatter such that data is encoded into the emitted ultrasonic backscatter. Encoding data in the form of different frequencies or frequency sequences, rather than in the form of different amplitudes, provides a method of ultrasonic data communication that is more protected from passive reflections and other reflective interferences.

The use of ultrasonic waves to operate and power an implantable device can be advantageous over other approaches because biological tissues have significantly lower absorption rates of ultrasonic waves than other types of waves such as RF waves. This property of ultrasonic waves can allow the device to be implantable at greater depths in the subject as well as to reduce tissue heating due to energy absorbed by the tissue.

As used herein, the singular forms “a,” “an,” and “the” include the plural reference unless the context clearly dictates otherwise.

Reference to “about” or “approximately” a value or parameter herein includes (and describes) variations that are directed to that value or parameter per se. For example, description referring to “about X” includes description of “X.”

It is understood that aspects and variations of the invention described herein include “consisting” and/or “consisting essentially of” aspects and variations.

Where a range of values is provided, it is to be understood that each intervening value between the upper and lower limit of that range, and any other stated or intervening value in that stated range, is encompassed within the scope of the present disclosure. Where the stated range includes upper or lower limits, ranges excluding either of those included limits are also included in the present disclosure.

It is to be understood that one, some or all of the properties of the various embodiments described herein may be combined to form other embodiments of the present invention. The section headings used herein are for organizational purposes only and are not to be construed as limiting the subject matter described.

Features and preferences described above in relation to “embodiments” are distinct preferences and are not limited only to that particular embodiment; they may be freely combined with features from other embodiments, where technically feasible, and may form preferred combinations of features. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

The disclosures of all publications, patents, and patent applications referred to herein are each hereby incorporated by reference in their entireties. To the extent that any reference incorporated by reference conflicts with the instant disclosure, the instant disclosure shall control.

As further described herein, a device for communicating via ultrasonic waves can include an ultrasonic transducer, a switch, and a circuit (e.g., an integrated circuit) configured to operate the switch. The ultrasonic transducer can receive ultrasonic waves (for example, ultrasonic waves transmitted by a separate device, such as an interrogator) and emit ultrasonic backscatter (which may be received, for example, by the interrogator). Without intervention, the frequency of the ultrasonic backscatter depends on the frequency of the incident ultrasonic waves. The switch, however, is configured to modulate the frequency of the emitted ultrasonic backscatter. For example, the switch may short the ultrasonic transducer at a desired frequency, which causes modulation of the ultrasonic backscatter frequency. The circuit can operate the switch to encode data in the emitted ultrasonic backscatter based on the frequency. For example, different frequencies may be used to communicate different bit patterns.

The data transmitted by the device may be information about the device, for example a device status, or data collected by the device (e.g., sensor information, such as information indicating an electrophysiological signal, a pH, a temperature, a pressure, or other physiological information sensed by one or more sensors of the device). According to some embodiments, the implantable device can include two or more electrodes that can detect an electrical pulse in a subject, for example an electrophysiological pulse transmitted by a nerve. According to some embodiments, the implantable device can include two or more electrodes that can emit an electrical pulse to a tissue of the body.

According to some embodiments, an implantable device can include an ultrasonic transducer capable of receiving ultrasonic waves emitted by an interrogator, converting the mechanical energy of the received ultrasonic waves into electrical energy to power the implantable device, and emitting ultrasonic backscatter to the interrogator. The interrogator may comprise one or more transducers for transmitting the ultrasonic waves and receiving the ultrasonic backscatter. According to some embodiments, data can be encoded into the ultrasonic backscatter by modulating a frequency of the ultrasonic backscatter. Encoding data into ultrasonic backscatter in this way allows for low-noise transmission through relatively noisy environments (such as within a body) compared to communication protocols that rely on amplitude modulation for encoding data into the ultrasonic backscatter. For example, encoding data using amplitude modulation suffers from noise issues such as passive reflections that can alter amplitudes of ultrasonic backscatter. However, encoding data using frequency modulation cannot be altered by passive reflections and therefore eliminates a noise factor and eliminates a need to calibrate the frequency modulation to account for such environmental noise. According to some embodiments, data encoded into the ultrasonic backscatter can be related to an instantaneous power hitting an ultrasonic transducer of the implantable device. According to some embodiments, data encoded into the ultrasonic backscatter can be related, for example, to sensor data from one or more sensors of the implantable device, any acknowledgment from the implantable device, and/or an operating status of the implantable device. The one or more sensors can be configured to detect a power level, pH, a temperature, a pressure, an electrophysiological pulse, and an analyte concentration (such as an oxygen level). Accordingly, the sensor data of the one or more sensors may include a power level, pH, a temperature, a pressure, an electrophysiological pulse, and an analyte concentration (such as an oxygen level).

The implantable device can include a switch for modulating a frequency of the ultrasonic backscatter for encoding data into the ultrasonic backscatter emitted from the implantable device. The ultrasonic backscatter can comprise a pattern that is unique to the implantable device. According to some embodiments, the pattern can comprise a signature of the implantable device that can be used to distinguish the implantable device from other devices.

According to some embodiments, data can be encoded into ultrasonic backscatter by selecting a switching frequency of the switch of the implantable device. The switching frequency may be selected from a plurality of different switching frequencies and each switching frequency may be associated with a predetermined bit pattern. A number of digital bits in any one of the predetermined bit patterns may be based on a binary logarithm of a number of switching frequencies of the plurality of different switching frequencies. The binary logarithm can be used to calculate a number of digital bits needed to encode a message. For example, if there are sixteen different switching frequencies that can be generated, then the binary logarithm of sixteen (which is four) specifies that given sixteen different switching frequencies, four digital bits can be communicated per transmission. By operating the switch to modulate a frequency of the ultrasonic backscatter based on the selected switching frequency, data associated with the selected switching frequency can be encoded into the ultrasonic backscatter. According to some embodiments, the switching frequency may be a sequence of different frequencies.

According to some embodiments, at least four different switching frequencies are available. For example, between 4 and 256 different switching frequencies are available (for example, between 4 and 8, between 8 and 16, between 16 and 32, between 32 and 64, between 64 and 128, or between 128 and 256 different switching frequencies are available).

According to some embodiments, the selection of the switching frequency may be based on one or more of sensor data, an intensity of the ultrasonic backscatter at a given frequency, an operating status of the implantable device. According to some embodiments, an operating status of the implantable device may indicate, for example, that the implantable device is powered ON by ultrasonic waves from an interrogator, that the interrogator has queried the implantable device for data and the implantable device is responding to the query, that a measurement conducted by the implantable device is complete, that a fault in the implantable device has been detected, that the implantable device is receiving a particular power level from the interrogator.

According to some embodiments, the switching frequency is a frequency at which the switch of the implantable device shorts the ultrasonic transducer of the device. For example, the implantable device can include the switch and a digital logic component that determines a state of the switch (for example, whether the switch should be open or closed). According to some embodiments, the closed state can be a shorted stated. A switching frequency can be selected by switching the switch between an open state and a closed state at the selected switching frequency. According to some embodiments, when the switch is in a closed state, the implantable device is unable to harvest energy (for example, to store in an energy storage circuit) from incoming ultrasonic waves because no energy flows into the implantable device. In the closed state, the implantable device can be configured such that at least some energy from the incoming ultrasonic waves is redirected. According to some embodiments, when the switch is in open state, the implantable device can be in a loaded configuration in which electrical energy is harvested from the ultrasonic waves and a maximum power is transferred from the ultrasonic waves to the implantable device to power the implantable device. The switching between the open and closed state of the switch can be configured to periodically (e.g., sinusoidally) vary acoustic energy reflected by the implantable device.

According to some embodiments, the ultrasonic backscatter is emitted at one frequency and that one frequency can provide a plurality of bits of information by encoding the plurality of bits into the one frequency. As described above, data such as the plurality of bits can be encoded into the one frequency by selecting a switching frequency of the implantable device that is associated with the plurality of bits and using the selected switching frequency to modulate the one frequency emitted from the implantable device. For example, Table 1 below lists examples of different backscatter frequency symbols each of which is associated with a different bit pattern. According each backscatter frequency and corresponding bit pattern is associated with a switching frequency. In the example of Table 1, the number of possible backscatter frequency symbols to select is sixteen and therefore the number of digital bits per interrogation can be four. According to some embodiments, the possible number of backscatter frequency symbols may be up to 32 or 64.

TABLE 1
Example bit patterns associated with different switching frequency

| Bit pattern | Frequency |
|---|---|
| 0000 | Frequency 0 |
| 0001 | Frequency 1 |
| 0011 | Frequency 2 |
| 0010 | Frequency 3 |

In the example of Table 1, the association between bit patterns to frequency is gray coded, so that adjacent frequencies only differ by 1 bit. Alternative coding schemes are also possible, with the optimal scheme depending on the typical systematic errors of a frequency detector.

The selected switching frequency may be selected from a plurality of predetermined switching frequencies, which may be based on the frequency of the ultrasonic waves received by the implantable device. For example, in some embodiments, the plurality of predetermined switching frequencies are within about 10%, within about 15%, within about 20%, within about 25%, or within about 30% of the frequency of the ultrasonic waves received by the implantable device.

According to some embodiments, the ultrasonic backscatter encoded with data can be emitted from the implantable device when the implantable device is powered up from ultrasonic waves received from an interrogator. The ultrasonic backscatter emitted from the implantable device can be received and decoded at the interrogator. According to some embodiments, the interrogator can be configured to analyze the ultrasonic backscatter to determine a frequency or changes in frequency of the ultrasonic backscatter. According to some embodiments, the interrogator can be configured to determine the switching frequency of the switch of the implantable device. Determining can include computing the switching frequency. Based on the determined switching frequency, the interrogator can be configured to associate the switching frequency with a pre-determined digital bit pattern to decode the encoded data in the ultrasonic backscatter. According to some embodiments, the interrogator may be pre-programmed to associate the determined switching frequency with a corresponding pre-determined digital bit pattern to decode the ultrasonic backscatter. According to some embodiments, the implantable device may be pre-programmed with one or more pluralities of different switching frequencies and each switching frequency may be associated with a different predetermined bit pattern. Likewise, the interrogator may be pre-programmed with the one or more pluralities of different switching frequencies for decoding the ultrasonic backscatter.

According to some embodiments, decoding the ultrasonic backscatter can comprise a bit error rate of up to 6%. According to some embodiments, decoding the ultrasonic backscatter can comprise a bit error rate of up to 5%. According to some embodiments, decoding the ultrasonic backscatter can comprise a bit error rate of up to 4%. According to some embodiments, decoding the ultrasonic backscatter can comprise a bit error rate of up to 3%. According to some embodiments, decoding the ultrasonic backscatter can comprise a bit error rate of up to 2%.

The ultrasonic communication between the implantable device and the interrogator can allow for closed loop communication. That is, the interrogator may change or adjust a parameter of the outgoing ultrasonic waves in response to the encoded data received from the implantable device and decoded at the interrogator. For example, depending on a power level received at the implantable device, a frequency of the emitted ultrasonic backscatter may be associated to a power level received at the implantable device. In this way, the implantable device can inform the interrogator how much instantaneous power is hitting the implantable device via the frequency of the emitted ultrasonic backscatter. In response, the interrogator can steer to adjust and track how well the ultrasonic waves from the interrogator are hitting that implantable device over time. Likewise, the implantable device may respond according to the ultrasonic waves received at the implantable device from the interrogator. A given response may include selecting a switching frequency based at least on instructions received at the implantable device from the interrogator. According to some embodiments, the instructions may cause the implantable device to execute one or more functions. The one or more functions may include one or more of a determination of average incident power received from the ultrasonic waves, detection of sensor information (such as an electrophysiology pulse, pH, oxygen level), and execution of a high sampling rate of sensor data stream such as an electrophysiological voltage time series (rather than pulse detection).

According to some embodiments, the implantable device and the interrogator may be programmed with a same plurality of different switching frequencies. For example, the implantable device may be programmed with a plurality of different switching frequencies from which one or more switching frequencies can be selected. Each switching frequency may be associated with a pre-determined bit pattern configured to communicate a message to an external device such as the interrogator. The interrogator may be configured to match a determined switching frequency with one of the plurality of different switching frequencies to determine the message from the implantable device. For example, should the determined switching frequency match a first switching frequency of the plurality of different switching frequencies, then the interrogator may be configured to interpret the pre-determined bit pattern of the first switching frequency as the message from the implantable device. According to some embodiments, for each plurality of different switching frequencies, the different pre-determined bit patterns associated with each different switching frequency are gray coded.

The implantable device can include one or more piezoelectric crystals that have different resonating operating modes. According to some embodiments, an optimal signal to noise ratio transmission from the implantable device to the interrogator can be achieved by modulating a frequency for the ultrasonic backscatter such that the frequency corresponds with a resonating operating mode of the one or more piezoelectric crystals. For example, a particular frequency of the ultrasonic backscatter may achieve a particular backscatter intensity receivable at the interrogator. The intensity of the particular backscatter intensity can be a relative maximum intensity. According to some embodiments, the particular frequency can correspond with a characteristic (such as a relative minimum) of impedance data of the one or more piezoelectric crystals. According to some embodiments, the backscatter intensity and one or more characteristics of the impedance data can be used to determine a process for selecting a set of switching frequencies that can modulate the frequency of the ultrasonic backscatter such that a

According to some embodiments, an implantable device can include an ultrasonic transducer configured to receive ultrasonic waves from an interrogator and emit ultrasonic backscatter. The implantable device may also include a switch configured to modulate a frequency of the ultrasonic backscatter and a controller such as an application-specific integrated circuit (ASIC) configured to communicate data to the interrogator using ultrasonic backscatter. In general, there is a finite amount of incoming acoustic energy originating from the interrogator which is then reflected by the implantable device back towards the interrogator. When an ultrasonic transducer (such as a piezoelectric circuit) of the implant is shorted, a maximum amount of the incoming acoustic energy is reflected. By adding a matched load to the piezoelectric circuit of the implantable device, electrical energy is harvested, and a maximum power can be transferred to the ASIC. According to some embodiments, the matched load can be a resistor.

Due to conservation of energy, switching the load changes the reflected energy. Traditionally, the change in reflected energy can be detected and enables digital communication by having a high amplitude reflected energy represent a 1 and low amplitude reflected energy (when energy is being harvested by the ASIC) represent a 0. Although the traditional amplitude modulation communication scheme works to transmit 1s and 0s, there are limitations due to environmental interference and low data rate between the implant and the interrogator. Alternatively, devices and systems described herein are configured to adjust the reflected energy by modulating a frequency of the reflected energy based on a frequency at which the piezoelectric circuit is shorted.

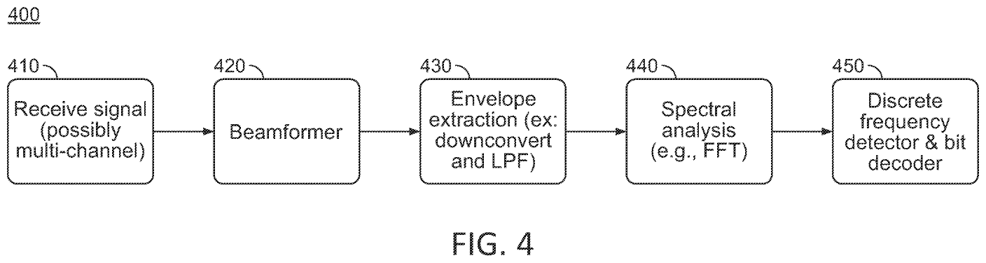

Rather than through individual high and low amplitude 0s and 1s, the devices and systems described herein communicate by altering the frequency of the load changes at the piezoelectric circuit such that multiple data bits can be communicated via frequency modulation ultrasonic backscatter. For example, an ultrasound signal can be transmitted from the interrogator to the piezoelectric circuit at 1.5 MHz. The piezoelectric circuit can then be switched at some switching frequency between a shorted configuration and a loaded configuration such that the reflected acoustic energy varies periodically (e.g., sinusoidally). At the interrogator, rather than detect a modulated amplitude at some threshold, a frequency analysis can be done, and the switching frequency of piezoelectric circuit can be estimated by analyzing the ultrasonic backscatter signal as shown in FIG. 4. The switching frequency can represent a set number of bits which can be recorded as data transferred for the interrogation.

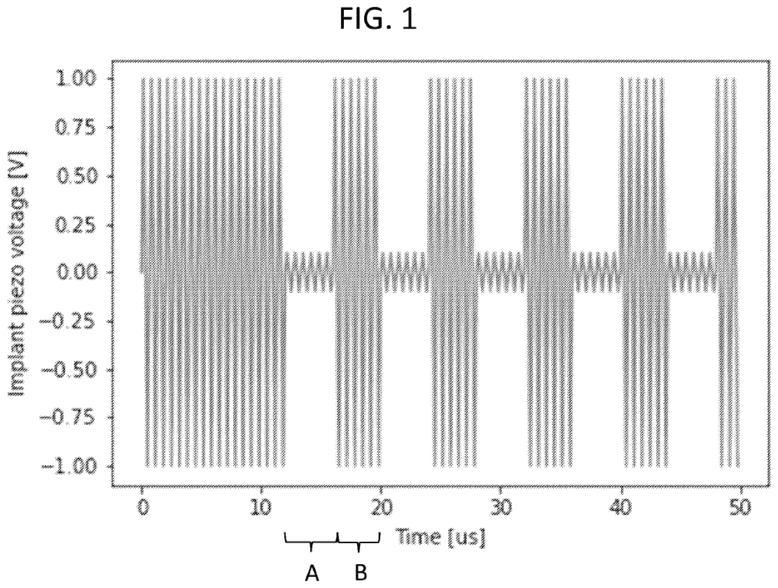

FIG. 1 shows an example of how a switch of the implantable device can operated to modulate the frequency of the ultrasonic backscatter, according to some embodiments. FIG. 1 shows a plot of switching behavior of the switch with amplitude (voltage) on the y axis and time (in microseconds) on the x axis. This figure represents the voltage on the piezoelectric crystal of the implantable device as a result of the switching frequency applied by the ASIC. The pattern on the piezoelectric crystal is reflected in the ultrasonic backscatter emitted from the implantable device In the example of FIG. 1, the carrier frequency is 1.5 MHZ and the switch is configured to short the implantable device at a frequency of 125 kHz. When the switch is closed, for instance, in region A, an emitted ultrasonic backscatter intensity has a minimal amplitude, which is indicative that a piezoelectric crystal of the implantable device is shorted. When the switch is open, for instance in region B, the emitted backscatter intensity is at a maximum amplitude. The ultrasonic backscatter received by the interrogator, which can identify the 125 kHz frequency segment, and can decode (for example, using a lookup table) a bit pattern associated with the 125 KHz frequency.

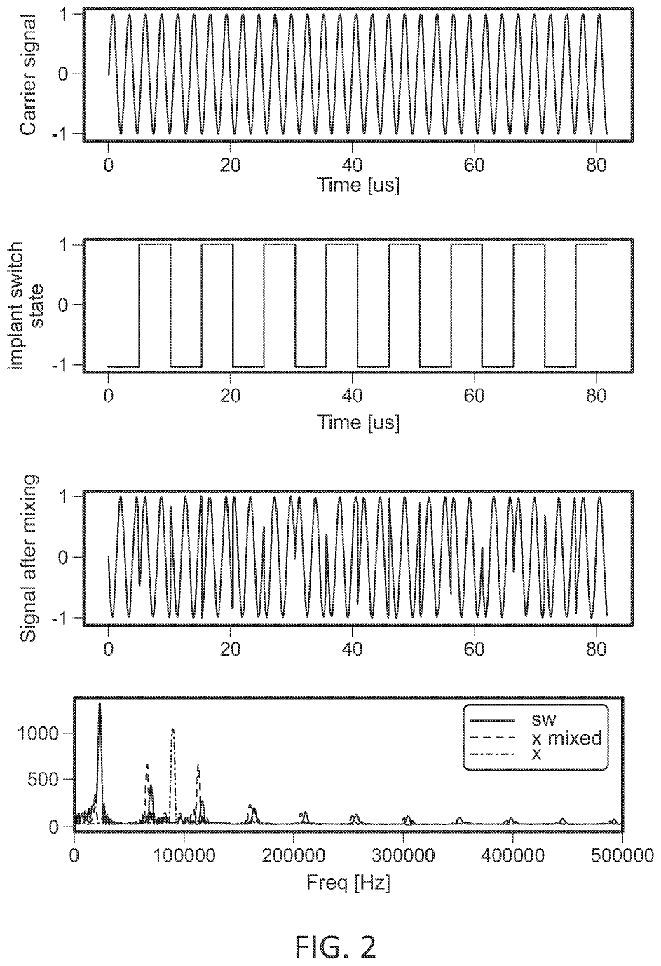

Another example of modulating a frequency of ultrasonic backscatter based on a frequency of the carrier signal and a switching frequency of a switch is provided in FIG. 2. FIG. 2 shows what happens to a carrier signal (top panel) at the implantable device in the frequency domain based on a switch state (second panel) of an implantable device. Specifically, the top panel represents an incoming signal that would be an input to the implantable device. The second panel (“implant switch state”) represents a state of a switch of the implantable device that is determined by a digital logic controller configured to operate the switch. Examples of the digital logic controller can be an application specific integrated circuit (ASIC), microcontroller, field programmable gate array (FPGA), or other similar components. Specifically, the second panel shows that the switch is being operated to go between two states a particular switching frequency. The particular switching frequency of the switch affects how the ultrasonic backscatter (reflected acoustic energy) is modulated. The third panel (“signal after mixing”) shows an example of an ultrasonic backscatter signal generated from mixing the carrier signal (top panel) with the switch state (second panel). By the switching the switch at the switching frequency, a frequency of the ultrasonic backscatter signal is modulated. Such frequency modulation may be more robust than amplitude modulation since amplitude modulation can be corrupted with passive environmental interferences.

The bottom panel of FIG. 2 shows a different representation of the ultrasonic backscatter signal shown in panel the third panel. The plot shown in the bottom panel may be referred to as the frequency domain and illustrates that different frequency content can be created by multiplying the carrier signal and the switch state signal together. In the example shown in the bottom panel, the frequency of the switch is the low-frequency peak (“sw”) and the frequency of the carrier signal is a higher frequency (“x”). By multiplying the frequency of the switch and the frequency of the carrier signal, a new frequency content can be generated and is shown in orange (“x mixed”). After mixing (the orange signal in the bottom panel), the frequency of the ultrasonic backscatter signal is “mixed” or shifted by + and − the switching frequency. The frequency content of the ultrasonic backscatter signal received at the interrogator remains centered at 1.5 MHz, but the amplitude of the backscatter varies with some frequency. In this case, even if some amplitude changes are not detected due to typical amplitude modulation issues, the frequency can be detectable.

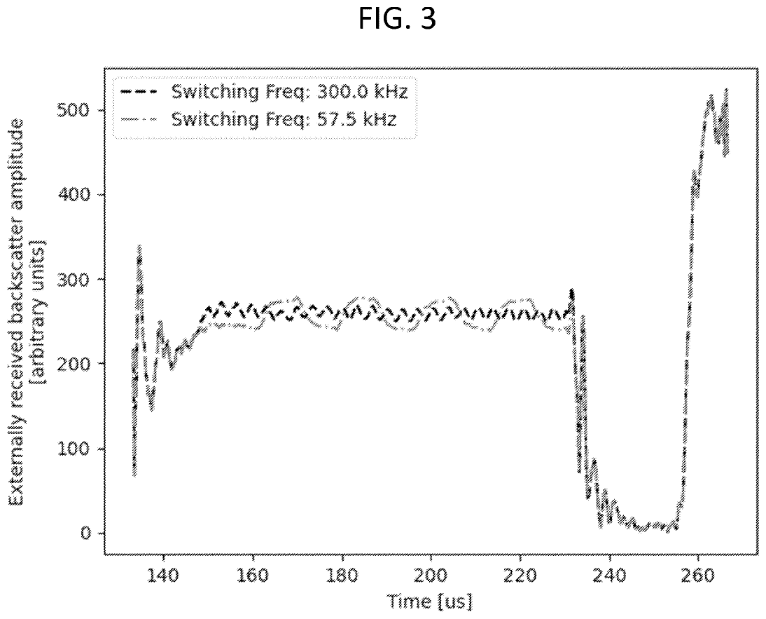

According to some embodiments, digital bits can be encoded by associating a particular bit (or bit pattern) to a backscatter frequency or (more preferably) to a switching frequency of the switch. For example, suppose an implantable device can backscatter one of 16 different backscatter frequencies as shown in Table 1 above. FIG. 3 shows an example of envelope-extracted ultrasonic backscatter at different switching frequencies, represented by Frequency 0 (300.0 kHz) and Frequency 1 (57.5 kHz) of Table 1, according to some embodiments. The y axis is an intensity that is proportional to the ultrasonic pressure that is received by the interrogator and the x axis is time (in microseconds). The curves of FIG. 3 represent backscatter frequencies receivable at the interrogator from the implantable device. Comparing the two frequencies shown, Frequency 0 is a relatively high frequency and Frequency 1 is a relatively low-frequency oscillation.

FIG. 4 shows a flow chart that describes a method 400 for detecting a signal at the interrogator, according to some embodiments. The method 400 can be applied for receiving different frequencies and parsing apart the different frequencies. At step 410, the interrogator generates a signal. The signal, if it's a multi-element transducer, can be focused at a particular point for the beam former. At step 420, the original signal or focused signal can be processed at the beam former. The beam former can be used to emphasize a certain spatial region of the receiving signal. At step 430, envelope extraction off of the receiving signal can be done. At step 440, a spectral analysis of the envelope extraction can be done. The spectral analysis can include a frequency breakdown of the receiving signal into different frequency components. An example of such spectral analysis includes fast Fourier transformations. At step 450, based on the output of the spectral analysis, a frequency can be determined.

According to some embodiments, an interrogator includes an array of ultrasonic transducers. The array of transducers can include channels configured to steer focusing of ultrasonic waves and steer from where ultrasonic backscatter signals are received. Each channel is signal path comprising physical elements (electronic components) of the transducer and interrogator. Each channel can receive different information, for example, based on where each channel is physically located on the transducer array. Each channel can also can transmit different signals which lets the beam focus at different points. In this way, multiple channels can allow for signal diversity when you have a multi-element system and you can choose to combine those signals however it makes sense. For example, depending on an angle or distance of the implantable device from the transducer array, different chatter can be received on each of the different channels. The different chatter can be used to piece together a single picture of the communication received from the implantable device.

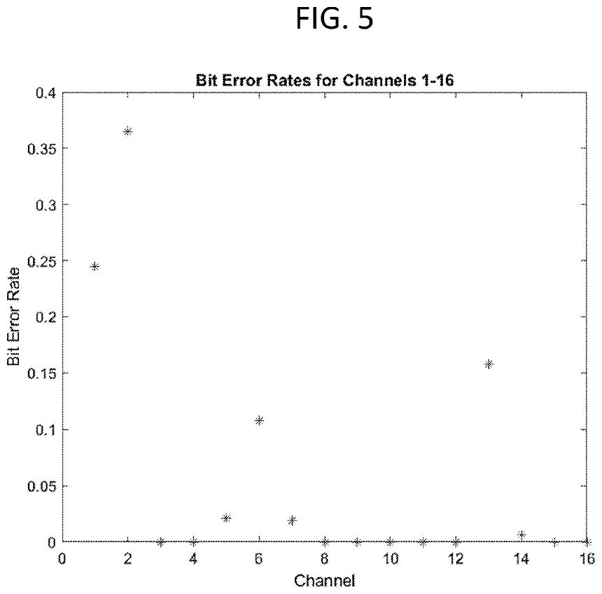

FIG. 5 shows a plot of bit error rate (BER) for each transducer channel of an interrogator that includes sixteen channels, according to some embodiments. The y-axis is a scale from 0 to 1 and the x-axis is an array of the channels of the transducer at the interrogator. The different channels of the interrogator are configured to receive ultrasonic backscatter at different positions in space. In the example of FIG. 5, a 16-element transducer was used to receive ultrasonic backscatter. According to some embodiments, the ultrasonic backscatter can be acquired simultaneously across all channels, and thus variation in performance across the transducer array can be due to spatial variation in the backscatter characteristics. BER can be calculated by counting a number of received bits that do not match expected bits. The low BER shown in FIG. 5 indicate that a majority of bits decoded were decoded correctly. An example of correct decoding is a zero bit is sent to the interrogator and a zero bit is received by the interrogator and decoded as a zero bit.

Overall, FIG. 5 shows an example having relatively few errors in the ultrasonic communication protocol with some channels (such as channel 1 and channel 2) having a relatively higher bit error rate. According to some embodiments, the relative bit error may be due to a physical location of those elements relative to the implant. According to some embodiments, a signal quality can be based on a spatial geometry of the transducers and channels.

Despite channel 2 having a relatively higher bit error rate compared to the other channels, one or more channels that are relatively low in bit error rate can be selected to get a view of the communication signal from other channels. According to some embodiments, to compensate for one or more channels that have a relatively high bit error rate, in a multi-element system you can select one or more other channels based on a signal quality criteria. According to some embodiments, to compensate for one or more channels that have a relatively high bit error a beam former can be used to combine information from all channels into a weighted average signal. The term “relatively high bit error rate” is intended to be relative to certain channel/frequency with no or very low bit error rates, and is not intended to indicate that the bit error rate of those channels is unusable or otherwise impairs the communication.

According to some embodiments, advantages of the ultrasonic communication described herein includes low error rate (see FIG. 5) and low power that enable effective closed loop communication. Closed loop communication can be used for powering implantable devices that may be implanted deep in the body. According to some embodiments, depending on an input voltage to the piezoelectric crystal of the implantable device, the implantable device can backscatter. In this way, if an interrogator configured to determine how much power is hitting the implantable device can conduct electronic steering to compensate or adjust the power depending on requirements of the implantable device. Thus, a closed loop communication can include transmitting power via ultrasonic waves from the interrogator to the implantable device, emitting from the implantable device ultrasonic backscatter, and adjusting a frequency of the ultrasonic backscatter based on a voltage level input at the implantable device. An example of a closed loop communication include charging the implantable device at a target rate based on a charge level of the implantable device.

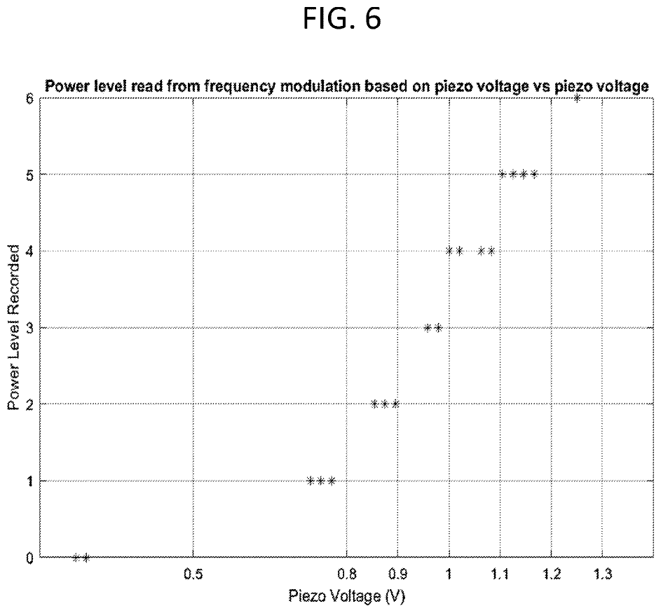

Frequency modulation of ultrasonic backscatter can be applied to communicate information about variations in piezoelectric voltage at the implantable device. Variation in the piezoelectric voltage indicate fluctuations in input power (current×voltage). Such variations can occur in a hand-held ultrasound link. Table 2 below shows exemplary voltage range readings that can be associated with a specific switching (or shorting) frequency of the piezoelectric at the implantable device. At the interrogator, the ultrasonic backscatter can be received and decoded by associating the frequency of the ultrasonic backscatter and/or the switching frequency at the implantable device to a predetermined bit pattern as explained above. Each bit pattern can represent a power level that can be interpreted by the interrogator. For example, if the implantable device has an input voltage less than half a volt, then the implantable device may set the switching frequency to be 50,000 Hz. The interrogator can be configured to calculate the switching frequency and if it calculates that the switching frequency is anything less than 55,000, then the interrogator can record that the voltage on the piezoelectric on the implantable device was less than half a volt and for discretization it can be referred to power level zero. FIG. 6 shows an exemplary plot of power levels read from frequency modulation based on piezoelectric voltage at the implantable device using the thresholds of Table 2. Such data and correlations as shown in Table 2 and FIG. 6 can be used to effectively charge the implantable device based on its charging needs.

TABLE 2
Example associations between voltage,
frequency, and power levels

| Voltage Read | Frequency | Frequency Read | Power Level |
|---|---|---|---|
| Cutoffs | Set | Cutoffs (Hz) | Recorded |
| Vₒᵤₜ < 0.5 V | 50000 | <55000 | 0 |
| 0.5 V < Vₒᵤₜ < 0.8 V | 68181 | 55000 < f < 70000 | 1 |
| 0.8 V < Vₒᵤₜ < 0.9 V | 93750 | 70000 < f < 100000 | 2 |
| 0.9 V < Vₒᵤₜ < 1 V | 125000 | 100000 < f < 130000 | 3 |
| 1 V < Vₒᵤₜ < 1.1 V | 150000 | 130000 < f < 170000 | 4 |
| 1.1 V < Vₒᵤₜ < 1.2 V | 187500 | 170000 < f < 200000 | 5 |
| 1.2 V < Vₒᵤₜ < 1.3 V | 250000 | 200000 < f < 300000 | 6 |
| Vₒᵤₜ > 1.3 V | 375000 | >300000 | 7 |

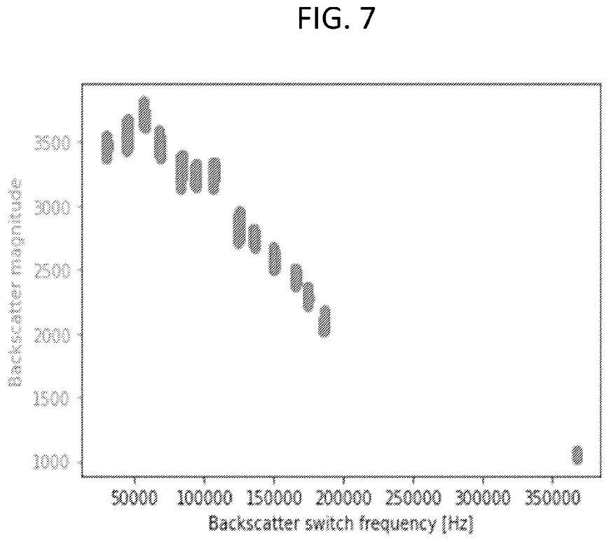

According to some embodiments, plotting a magnitude of the ultrasonic backscatter frequencies versus the calculated switching frequency can show a trend that may be indicative of a physical property of the piezoelectric at the implantable device. FIG. 7 shows an example of ultrasonic backscatter magnitudes received at the interrogator versus the calculated switching frequency for a channel, according to some embodiments. As shown in this figure, different switching frequencies have higher or lower signal-to-noise, likely depending on the physical properties of the ultrasonic transducer (e.g., the piezoelectric crystal) used.

According to some embodiments, the switching frequency can be determined by a controller of the implantable. The determination can be random or can be based on a characteristic of the piezoelectric at the implantable device. For example, suppose a high magnitude of ultrasonic backscatter at the interrogator is desired. In the example of FIG. 7, there is a relative maximum in backscatter magnitude around 60,000 Hz. Thus, to achieve a high magnitude of ultrasonic backscatter at the interrogator, the switching frequency may be selected to be with a region that includes a relative maximum backscatter magnitude. That is, depending on an application, the switching frequency may be selected based at least partially on a magnitude of the ultrasonic backscatter at the interrogator.

According to some embodiments, a switch frequency that is near a resonance property of the piezoelectric crystal of the implantable device can potentially result in a relative maximum backscatter at the detectors. According to some embodiments, a bigger ultrasonic backscatter magnitude at the detectors can be lead to a lower bit error rate. According to some embodiments, the implantable device may be calibrated to select a switching frequency based on a magnitude received at the interrogator and this selected switching frequency may correspond to particular regions of an impedance spectrum of the piezoelectric.

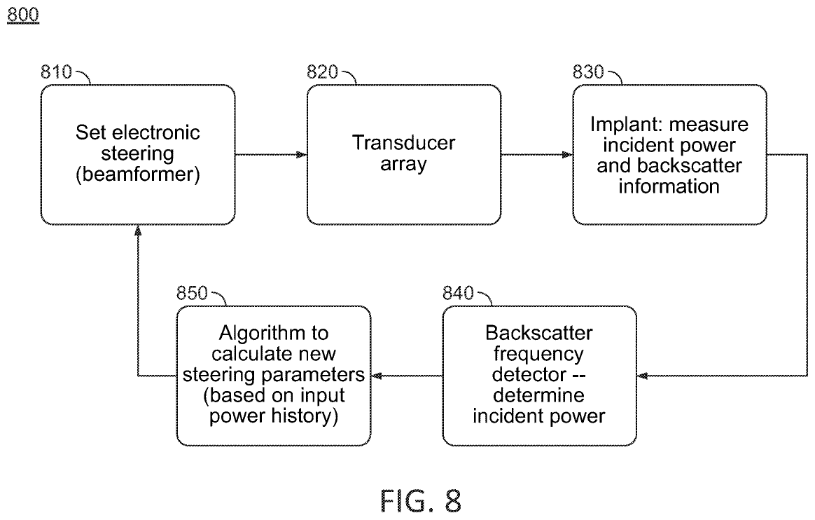

According to some embodiments, a closed-loop power-delivery algorithm can be implemented using the communication protocol described herein. FIG. 8 shows an example flow diagram that describes a steering method 800 for closed-loop communication, according to some embodiments. Method 800 outlines processing of power level data in a closed looped system. That is, method 800 describes how power level data can be communicated.

At step 810, an electronic steering can be set at the interrogator. At step 820, the electronic steering is communicated with the transducer array at the interrogator. The transducer array can emit ultrasonic waves to the implantable device. At step 830, the implantable device can receive the ultrasonic waves, measure incident power, and emit ultrasonic backscatter containing information. At step 840, the one or more ultrasonic transducers of the interrogator can receive the ultrasonic backscatter from the implantable device and a processor of the interrogator can decode the ultrasonic backscatter to determine incident power received at the implantable device. At step 850, at the interrogator, new steering parameters can be calculated based on the determined incident power received at the implantable device.

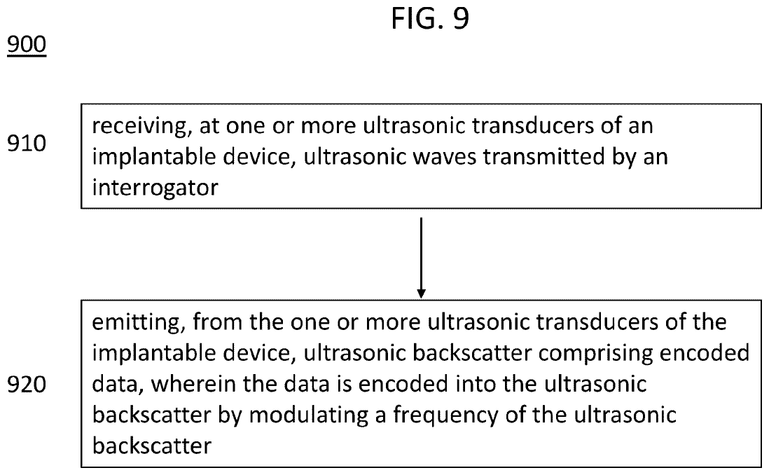

FIG. 9 shows a flow diagram that describes a method 900 for low-power communication using ultrasonic waves, according to some embodiments. The method 900 may be implemented by an implantable device as described herein.

At step 910, one or more ultrasonic transducers of an implantable device may receive ultrasonic waves transmitted by an interrogator. The ultrasonic waves may be transmitted by one or more ultrasonic transducers of the interrogator.

At step 920, the one or more ultrasonic transducers of the implantable device may emit ultrasonic backscatter comprising encoded data. The data can be encoded into the ultrasonic backscatter by modulating a frequency of the ultrasonic backscatter.

According to some embodiments, the implantable device may include a switch configured to open and close a piezoelectric circuit of the implantable device. According to some embodiments, when the switch is closed, the circuit is shorted. A controller can be configured to operate the switch between an open state and a closed state and select a switching frequency at which the switch changes between the open state and the closed state. According to some embodiments, the method 900 may include selecting the switching frequency from a plurality of different switching frequencies. As described further above, each switching frequency can be associated with a pre-determined bit pattern that can comprise a plurality of bits. Associations between each switching frequency and the pre-determined bit pattern can be gray coded.

According to some embodiments, the implantable device can include one or more sensors configured to detect sensor information such as one or more of a power level, pH, a temperature, a pressure, an electrophysiological pulse, and an analyte concentration (such as an oxygen level). According to some embodiments, method 900 can include detecting sensor information by the one or more sensors of the implantable device. The implantable device can include a status indicator configured to determine an operating status of the implantable device. The operating status can include, for example, whether the implantable device is ON, OFF, in measuring mode, functioning well, has error encountered, is fully charged, or requires more power to reach a full charge level. According to some embodiments, the selection of the switching frequency can be based on one or more of instructions from the interrogator, sensor information detected by one or more sensors of the implantable devices, and the operating status of the implantable device.

According to some embodiments, the method 900 can include decoding the ultrasonic backscatter at the interrogator. The decoding can include analyzing an incoming ultrasonic backscatter to compute a frequency of the ultrasonic backscatter and a switching frequency of the switch of the implantable device. The computed switching frequency can be matched to a predetermined bit pattern, which may be recorded as data transferred from the device.

According to some embodiments, the implantable device can include one or more first electrodes configured to emit an electrical pulse to a tissue. According to some embodiments, the implantable device can include one or more second electrodes configured to detect an electrophysiological pulse. According to some embodiments, method 1000 can include emitting an electrical pulse and detecting an electrophysiological pulse.

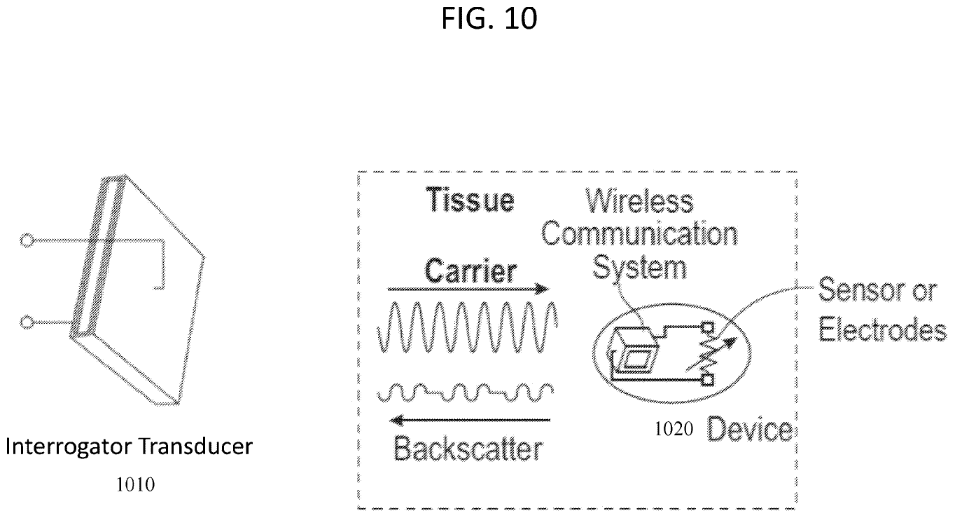

FIG. 10 shows an interrogator 1010 in communication with an implantable device 1020. The external ultrasonic transceiver emits ultrasonic waves (“carrier waves”), which can pass through tissue. The carrier waves cause mechanical vibrations on the ultrasonic transducer (e.g., a bulk piezoelectric transducer, a PMUT, or a CMUT). A voltage across the ultrasonic transducer is generated, which imparts a current flowing through a circuit (e.g., an integrated circuit) on the implantable device. The current flowing through to the ultrasonic transducer causes the transducer on the implantable device to emit backscatter ultrasonic waves. In some embodiments, the circuit modulates the frequency of the ultrasonic transducer to encode information by shorting the circuit at a switching frequency, and the resulting ultrasonic backscatter waves encode the information. The backscatter waves can be detected by the interrogator, and can be analyzed to interpret information encoded in the ultrasonic backscatter. According to some embodiments, the implantable device 1020 may include one or more sensors. According to some embodiments, the implantable device 1020 may include one or more electrodes.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

1. A method for communicating using ultrasonic waves comprising:
receiving, at one or more ultrasonic transducers of an implantable device, ultrasonic waves transmitted by an interrogator; and
emitting, from the one or more ultrasonic transducers of the implantable device, ultrasonic backscatter comprising encoded data, wherein the data is encoded into the ultrasonic backscatter by modulating a frequency of the ultrasonic backscatter.

2. The method of claim 1, comprising selecting a switching frequency from a plurality of different switching frequencies to modulate the frequency of the ultrasonic backscatter, wherein the switching frequency is configured to encode the data into the ultrasonic backscatter.

3. The method of claim 2, wherein each switching frequency is associated with a different predetermined bit pattern.

4. The method of claim 2 or 3, wherein modulating the frequency of the ultrasonic backscatter comprises switching a switch of the implantable device at the switching frequency.

5. The method of any one of claims 2-4, wherein each pre-determined bit pattern comprises two or more digital bits.

6. The method of any one of claims 2-5, wherein pre-determined bit patterns associated with the plurality of different switching frequencies are gray coded.

7. The method of any one of claims 2-6, wherein the switching frequency is selected based at least on instructions received from the interrogator via the ultrasonic waves.

8. The method of any one of claims 2-7, wherein the plurality of different switching frequencies is selected based on at least an intensity of the ultrasonic backscatter at a given frequency.

9. The method of any one of claims 1-8, comprising detecting sensor information from one or more sensors of the implantable device, wherein the data comprises the sensor information, and selecting the switching frequency from the plurality of different switching frequencies based at least on the sensor information.

10. The method of claim 9, wherein the sensor information comprises one or more of a power level, pH, a temperature, a pressure, an electrophysiological pulse, and an analyte concentration.

11. The method of claim 10, wherein the analyte concentration comprises an oxygen level.

12. The method of any one of claims 1-11, comprising determining operating status information of the implantable device, wherein the data comprises determined operating status information, and selecting the switching frequency from the plurality of different switching frequencies based at least on the determined operating status information.

13. The method of any one of claims 1-12, comprising selecting a sequence of two or more switching frequencies from a plurality of different switching frequencies to encode the data, wherein each switching frequency is associated with a different predetermined bit pattern.

14. The method of any one of claims 1-13, comprising:
receiving, at the interrogator, the ultrasonic backscatter comprising the encoded data;
decoding, at the interrogator, the ultrasonic backscatter received, wherein decoding the ultrasonic backscatter comprises analyzing an incoming frequency of the ultrasonic backscatter received at the interrogator to determine a computed switching frequency of a switch modulating the frequency of the ultrasonic backscatter, matching the computed switching frequency to a predetermined bit pattern, and recording the predetermined bit pattern as decoded data transferred from the implantable device.

15. The method of claim 14, wherein the decoded data has a bit error rate of up to 6%.

16. The method of any one of claims 1-15, comprising adjusting a parameter of the ultrasonic waves transmitted by the interrogator based on the ultrasonic backscatter received from the implantable device.

17. The method of any one of claims 1-16, wherein modulating the frequency of the ultrasonic backscatter comprising switching a switch of the implantable device between an open state and a closed state at a switching frequency from a plurality of different switching frequencies.

18. The method of claim 17, wherein the closed state shorts the one or more ultrasonic transducers of the implantable device.

19. The method of claim 17 or 18, wherein the open state is a loaded configuration in which electrical energy is harvested from the ultrasonic waves and a maximum power is transferred from the ultrasonic waves to the implantable device to power the implantable device.

20. The method of any one of claims 17-19, wherein switching the switch of the implantable device between the open state and the closed state is configured to periodically vary acoustic energy reflected by the implantable device.

21. The method of any one of claims 1-20, comprising transmitting ultrasonic waves from the interrogator to the implantable device, wherein the ultrasonic waves instruct the implantable device to execute one or more functions.

22. The method of claim 21, wherein the one or more functions comprises determining average incident power from the ultrasonic waves.

23. The method of any one of claims 1-20, comprising transmitting the ultrasonic waves from the interrogator to the implantable device.

24. The method of any one of claims 1-23, comprising receiving the ultrasonic backscatter at the interrogator.

25. The method of any one of claims 1-24, wherein the interrogator comprises one or more transducers for transmitting the ultrasonic waves and receiving the ultrasonic backscatter.

26. A device for communicating via ultrasonic waves, comprising:
an ultrasonic transducer configured to receive ultrasonic waves and emit ultrasonic backscatter;
a switch configured to modulate a frequency of the emitted ultrasonic backscatter; and
a circuit configured to operate the switch to encode data in the emitted ultrasonic backscatter based on the frequency.

27. The device of claim 26, wherein the switch is switchable at a switching frequency from a plurality of different switching frequencies and the data encoded in the emitted ultrasonic backscatter is based on the switching frequency, wherein each switching frequency is associated with a different predetermined bit pattern.

28. The device of claim 27, wherein each pre-determined bit pattern comprises two or more digital bits.

29. The device of claim 27, wherein pre-determined bit patterns associated with the plurality of different switching frequencies are gray coded.

30. The device of any one of claims 27-29, wherein the switching frequency is based at least on a relative maximum intensity of ultrasonic backscatter receivable at an interrogator.

31. The device of any one of claims 27-30, comprising one or more sensors configured to detect sensor information, wherein the data comprises detected sensor information, and the switching frequency from the plurality of different switching frequencies is based at least on the detected sensor information.

32. The device of claim 31, wherein the sensor information comprises a power level of the device.

33. The device of claim 31 or 32, wherein the one or more sensors comprises one or more of a pressure sensor, a pH sensor, a temperature sensor, and an analyte sensor.

34. The device of any one of claims 27-33, comprising a status indicator, wherein the switching frequency from the plurality of different switching frequencies is based at least on one or more statuses of the status indicator.

35. The device of any one of claims 27-34, the switch is switchable in a sequence of two or more switching frequencies from a plurality of different switching frequencies to encode the data.

36. The device of claim 35, wherein each switching frequency is associated with a different predetermined bit pattern.

37. The device of any one of claims 26-36, wherein the switch is switchable between an open state and a closed state at a switching frequency from a plurality of different switching frequencies.

38. The device of claim 37, wherein the closed state shorts the ultrasonic transducer.

39. The device of claim 37 or 38, wherein the open state is a loaded configuration in which electrical energy is harvested from the ultrasonic waves and a maximum power is transferred from the ultrasonic waves to the device to power the device.

40. The device of any one of claims 37-39, wherein switching the switch between the open state and the closed state is configured to periodically vary acoustic energy reflected by the device.

41. The device of any one of claims 26-40, wherein the device is an implantable device.

42. The device of any one of claims 26-41, comprising two or more electrodes configured to emit an electrical pulse to a tissue.

43. The device of any one of claims 26-42, comprising two or more electrodes configured to detect an electrophysiological pulse.

44. A system for communicating via ultrasonic waves, comprising:
the device of any one of claims 26-43; and
an interrogator comprising:
one or more ultrasonic transducers configured to receive the emitted ultrasonic backscatter and transmit the ultrasonic waves to the device; and
a processor configured to decode the emitted ultrasonic backscatter.

45. The system of claim 44, wherein an interrogator is configured to transmit the ultrasonic waves comprising instructions and the switching frequency is selected based at least on the instructions received by the device.

46. The system of claim 44 or 45, wherein the interrogator is configured to decode the ultrasonic backscatter by analyzing an incoming frequency of the ultrasonic backscatter to determine a computed switching frequency of the switch, match the computed switching frequency to a predetermined bit pattern, and record the predetermined bit pattern as data transferred from the device.

47. The system of claim 46, wherein decoding the ultrasonic backscatter results in a bit rate error of up to 6%.

48. The system of any one of claims 44-47, wherein the interrogator is configured to adjust a parameter of the ultrasonic waves transmitted by the interrogator based on the ultrasonic backscatter received from the device.

49. The system of any one of claims 44-48, wherein the ultrasonic wave transmitted from an interrogator are configured to instruct the device to execute one or more functions.

50. The system of claim 49, wherein the one or more functions comprises determining average incident power from the ultrasonic waves.
